(19) United States
(12) Patent Application Publication
(10) Pub. No.: US 2021/0077529 A1
(43) Pub. Date: Mar. 18, 2021

(54) T-CELL EXHAUSTION, METHODS & COMPOSITIONS RELATING THERETO

(71) Applicant: MEMORIAL SLOAN KETTERING CANCER CENTER, New York, NY (US)

(72) Inventors: Santosha Vardhana, New York, NY (US); Lydia Whitney Stillman Finley, New York, NY (US); Craig B. Thompson, New York, NY (US)

(21) Appl. No.: 17/048,248

(22) PCT Filed: Apr. 16, 2019
(86) PCT No.: PCT/US19/27610

§ 371 Date: Oct. 16, 2020

Related U.S. Application Data
(60) Provisional application No. 62/657,932, filed on Apr. 16, 2018.

(51) Int. Cl. A61K 35/17; A61K 31/198; C12N 5/0783

(52) U.S. Cl. CPC A61K 35/17; A61K 31/198; C12N 5/0636; C12N 2501/515; C12N 2501/2302; C12N 2502/70; C12N 2501/51; C12N 2502/1121

(57) ABSTRACT

The present invention provides methods for the induction and monitoring of T cell exhaustion in vitro. The methods are effective for both human and non-human T cells, and for both antigen-specific and polyclonal T cells. The present invention also provides methods to screen for and/or evaluate pharmacologic agents that can either induce or reverse T cell exhaustion. The present invention also provides certain agents that can reverse T cell exhaustion.

This application claims the benefit of priority of U.S. Provisional Patent Application No. 62/657,932 filed on Apr. 16, 2018, the content of which is hereby incorporated by reference in its entirety.

### STATEMENT OF GOVERNMENT SUPPORT

This invention was made with government support under grant number CA009207 awarded by the National Institutes of Health. The government has certain rights in the invention.

### INCORPORATION BY REFERENCE

For countries that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND TO THE INVENTION

In cancer and chronic infections, T cells are often exposed to antigens (such as tumor antigens or viral antigens) for prolonged periods of time. This prolonged chronic exposure to antigen can lead to a deterioration of T cell function referred to as “T cell exhaustion”—which is associated with a reduced ability of the immune system to control tumor growth and to control chronic infections. For a review of T cell exhaustion see: Wherry and Kurachi, “Molecular and Cellular Insights into T Cell Exhaustion,” Nature (2015), Vol. 15, pp. 486-499, the contents of which are hereby incorporated by reference. Exhausted T cells typically exhibit increased expression of inhibitory receptors (such as PD-1, LAG-3, and PD-L1), decreased production of effector cytokines, decreased proliferation rates, and decreased target cell killing activity. Revitalization of exhausted T cells can reinvigorate immunity. For example, inhibitors of PD1 and PD-L1 (so-called “immune checkpoint inhibitors”) can reverse T cell exhaustion and reinvigorate the response of the immune system to tumors.

Current methods for studying T cell exhaustion involve inducing the exhausted phenotype in vivo. The requirement to perform such studies in vivo presents a barrier to studying how T cell exhaustion works in the human immune system, and to performing screens for new therapeutic agents capable or modulating (either positively or negatively) T cell exhaustion. Accordingly, there is a need in the art for new and improved methods that can be used to induce and study T cell exhaustion in vitro, and to screen for new therapeutic candidates in vitro or study the effects of known or new therapeutic agents in vitro. The present invention addresses these and other needs.

### SUMMARY OF THE INVENTION

The present invention is based, in part, on a series of new methods and related new discoveries that are summarized below, and that are further described in the “Brief Description of the Figures,” “Figures,” Detailed Description,” “Examples,” and “Claims” sections of this patent disclosure.

The present invention provides new methods for the induction and monitoring of T cell exhaustion in vitro, providing significant advantages over prior methods that required T cell exhaustion to be induced in vivo. These new methods differ from classical methods of inducing T cell exhaustion—which typically rely on in vivo models of chronic viral infection or adoptive transfer of T cells into tumor-bearing mice followed by isolation of tumor-infiltrating lymphocytes. The new in vitro methods provided herein are effective with both human and non-human T cells, and with both antigen-specific and polyclonal T cells. Furthermore, the methods provided herein offer a mechanism by which to test whether T cells can be re-invigorated with pharmacologic agents in an in vitro system. For example, the in vitro methods provided herein can be used for pre-clinical evaluation of immunomodulatory agents, including, but not limited to, immune checkpoint inhibitors. The methods provided herein are also useful for prognostic applications—enabling the evaluation and measurement in vitro of the T cell exhaustion status of patient-derived cells and/or the capacity of patient-derived T cells to withstand exhaustion in response to chronic stimulation.

The present methods induce T cell exhaustion by in vitro stimulation of T cell receptors. For example, in one embodiment, the present invention provides a method of inducing T cell exhaustion in vitro, the method comprising: culturing T cells in vitro for a first time-period under conditions that acutely stimulate T-cell receptor signaling, and subsequently culturing the T cells in vitro for a second time-period under conditions that chronically stimulate T-cell receptor signaling, thereby generating exhausted T cells.

Importantly, the present methods can effectively induce T cell exhaustion by either generalized stimulation of TCRs on polyclonal T cells in a non-antigen specific manner, or by or antigen-specific stimulation of TCRs on antigen-specific T cells. In the former situation, generalized stimulation of TCRs on polyclonal T cells can be achieved by, for example, culturing the T cells with: (a) an anti-CD3 antibody and, optionally, an anti-CD28 antibody, or (b) with both an anti-CD3 antibody and an anti-CD28 antibody. In the latter situation, antigen-specific stimulation of TCRs on antigen-specific T cells can be achieved by, for example, culturing the antigen-specific T cells with antigen presenting cells, tumor cells, virus-infected cells, or other cells that present a peptide from the specific antigen on an MHC molecule on their surface (or by culturing the antigen-specific T cells with antigen presenting cells, tumor cells, virus-infected cells, or other cells in the presence of the specific antigen, or a peptide from the specific antigen, such that the antigen or peptide will be displayed on an MHC molecule on the cells' surface).

In some embodiments the T cells used (i.e. the starting/input population of T cells) in the methods of the present invention comprise, consist essentially of, or consist of, antigen-specific T cells. In some such embodiments the antigen-specific T cells are specific for a given epitope within the antigen of interest. In some such embodiments the antigen-specific T cells are monoclonal antigen-specific T cells. In some such embodiments the T cells used (i.e. the starting/input population of T cells) comprise, consist essentially of, or consist of, a substantially pure population of antigen-specific T cells. In some such embodiments the T cells used (i.e. the starting/input population of T cells) comprise, consist essentially of, or consist of, a substantially pure population of antigen-specific T cells specific for a given epitope within the antigen of interest. In some such embodiments the T cells used (i.e. the starting/input population of T cells) comprise, consist essentially of, or consist of, a substantially pure population of monoclonal antigen-specific T cells.

Such antigen-specific T cells can be used to generate antigen-specific exhausted T cells. For example, in one embodiment the present invention provides a method of inducing exhaustion of antigen-specific T cells in vitro, the method comprising: culturing antigen-specific T cells with either: (i) the antigen or peptide/epitope for which the T cells are specific and antigen-presenting cells, and optionally IL-2, or (ii) antigen-presenting cells that present the antigen or peptide/epitope for which the T cells are specific on an MHC molecule, and optionally IL-2, wherein the culturing is performed in vitro the for a first time-period, and subsequently culturing the T cells with: (i) the antigen or peptide/epitope for which the T cells are specific and antigen-presenting cells, tumor cells, or virus-infected cells, and optionally IL-2, or (ii) antigen-presenting cells, tumor cells, or virus-infected cells, that present the antigen or peptide/epitope for which the T cells are specific on an MHC molecule, and optionally IL-2, wherein the culturing is performed in vitro for a second time-period, thereby generating antigen-specific exhausted T cells.

In some embodiments the T cells used (i.e. the starting/input population of T cells) comprise, consist essentially of, or consist of, “non-antigen-specific T cells”—which are also referred to herein as “polyclonal T cells.” In some such embodiments the T cells used (i.e. the starting/input population of T cells) comprise, consist essentially of, or consist of, a substantially pure population of polyclonal T cells.

Such polyclonal T cells can be used to generate polyclonal exhausted T cells. For example, in one embodiment the present invention provides a method of inducing exhaustion of polyclonal T cells in vitro, the method comprising: (a) culturing polyclonal T cells with: (i) an anti-CD3 antibody and (ii) optionally an anti-CD28 antibody, and (iii) optionally IL-2, wherein the culturing is performed in vitro the for a first time-period, and (b) subsequently culturing the T cells with: (i) an anti-CD3 antibody, (ii) optionally an anti-CD28 antibody, and (iii) optionally IL-2, wherein the culturing is performed in vitro the for a second time-period, thereby generating polyclonal exhausted T cells.

The methods summarized above, and described elsewhere herein, all generate exhausted T cells—whether starting from antigen-specific T cells or polyclonal T cells. Typically the exhausted T cells will exhibit one or more of (or all of) the following characteristics: (a) increased expression of one of more of PD-1, LAG-3, and/or PD-L1, as compared to the starting T cells and/or non-exhausted T cells, (b) decreased production of one or more effector cytokines (such as IFNγ. TNFα and/or IL2) as compared to the starting T cells and/or non-exhausted T cells, (c) decreased proliferation as compared to the starting T cells and/or non-exhausted T cells, and (d) decreased target cell killing activity as compared to the starting T cells and/or non-exhausted T cells. In some embodiments the methods summarized above and described elsewhere herein comprise an step of performing an assay to measure one or more of (a) expression of PD-1, LAG-3, and/or PD-L1, (b) production of one or more effector cytokines, (c) proliferation rate, and/or (d) target cell killing activity in the cells generated using the method. This step is useful for assessing the exhaustion status of the cells. In some embodiments such an assay will be performed with “test” T cells and “control” T cells. The “test” T cells will typically be cells that have been subjected to a method of inducing exhaustion as described herein. In some embodiments the control T cells may be the starting/input population of T cells from which the test cells were generated. In some embodiments the control T cells may be T cells that have been subjected to only acute stimulation of T cell receptor signaling and not chronic stimulation of T cell receptor signaling (e.g. that have been subjected to the first step of the methods described herein but not the second step of such methods). Other suitable “test” and “control” cells can be selected as desired.

An important feature of the present methods is that they generate T cell exhaustion by prolonged in vitro stimulation of T cell receptors—whether that stimulation is a generalized stimulation of the TCRs on polyclonal T cells or antigen-specific stimulation of antigen-specific T cells. This prolonged stimulation is produced by culturing the T cells under a first set of conditions (that acutely stimulate T cell receptor signaling) for a first time period, and subsequently culturing the T cells under a second set of conditions (that chronically stimulate T cell receptor signaling) for a second time period. In some embodiments the first time-period is about 1-3 days (24-72 hours). In some embodiments the first time-period is about 1-2 days (24-48 hours). In some embodiments the first time-period is about 11/2-2 days (36-48 hours). In some embodiments the first time-period is about 2 days (48 hours). In some embodiments the second time-period is about 4-8 days (96-192 hours). In some embodiments the second time-period is about 6 days (144 hours). In some embodiments the second time-period is at least about 4 days (96 hours). In some embodiments the second time-period is at least about 6 days (144 hours). Typically during the second time period the he T cells may be re-plated or split or passaged or may have their culture media changed or supplemented several times. For example, in some embodiments, the T cells may be re-plated or split or passaged or may have their culture media changed or supplemented approximately every 1-2 days during the second time period. Typically, during the second time period the T cells will be re-plated or split or passaged or may have their culture media changed or supplemented several times—to provide a replenished supply of the generalized TCR stimulus (such as anti-CD3 and/or anti-CD28 antibodies). For example, in some embodiments, during the second time period the T cells will be re-plated or split or passaged or may have their culture media changed or supplemented approximately every 1-2 days.

In addition to providing methods for the generation of exhausted T cells, the present invention also provides populations of exhausted T cells obtained using the methods provided herein. In some such embodiments, the populations of exhausted T cells are substantially pure populations of exhausted T cells.

The present invention also provides a variety of methods for evaluating the effect of one more test agents on T cell exhaustion. For example, in one embodiment the present invention provides a method for evaluating the effect of one more test agents on T cell exhaustion, the method comprising: (a) performing a method to induce T cell exhaustion by in vitro stimulation of T cell receptors as described above or elsewhere herein, in the presence of a test agent(s) and in the absence of the test agent(s), and (b) performing an assay to measure one or more of: expression of PD-1, LAG-3, and/or PD-L1, production of one or more effector cytokines, proliferation rate, and/or target cell killing activity in the T cells produced in step (a), wherein if the T cells produced in the presence of the test agent exhibit increased expression of PD-1, LAG-3, and/or PD-L1, decreased production of the one or more effector cytokines, a decreased proliferation rate, or decreased target cell killing activity as compared to the T cells produced in the absence of the test agent, the test agent increases T cell exhaustion, and wherein: if the T cells produced in the presence of the test agent exhibit decreased expression of PD-1, LAG-3, and/or PD-L1, increased production of the one or more effector cytokines, an increased proliferation rate, or increased target cell killing activity as compared to T cells produced in the absence of the test agent, the test agent decreases T cell exhaustion.

Similarly, in another embodiment the present invention provides a method of evaluating the effect of one more test agents on reinvigoration of exhausted T cells, the method comprising: (a) contacting a first population of exhausted T cells produced using a method as described herein with one or more test agents to produce a test population of T cells, (b) contacting a second population of exhausted T cells produced using a method as described herein with either no test agent or with one or more control agents to produce a control population of T cells, (c) performing an assay to measure one or more of: expression of PD-1, LAG-3 and/or, PD-L1, production of one or more effector cytokines, proliferation rate, and/or target cell killing activity, in the test population of T cells and in the control population T cells, wherein: if the test population of T cells exhibits increased expression of PD-1, LAG-3, and/or PD-L1, decreased production of the one or more effector cytokines, a decreased proliferation rate, or decreased target cell killing activity as compared to the control population of T cells, the test agent decreases T cell reinvigoration, and wherein: if the test population of T cells exhibits decreased expression of PD-1, LAG-3, and/or PD-L1, increased production of the one or more effector cytokines, an increased proliferation rate, or increased target cell killing activity as compared to the control population of T cells, the test agent increases T cell reinvigoration.

In another embodiment the present invention provides a method of re-invigorating exhausted T cells, the method comprising contacting exhausted T cells with an effective amount of N-acetylcysteine. Similarly, in another embodiment the present invention also provides methods of reducing or reversing T cell exhaustion, such methods comprising contacting T cells with an effective amount of N-acetylcysteine. In addition, in yet another embodiment the present invention provides methods of stimulating or increasing a T cell-mediated immune response (e.g. to a tumor or to a chronic infection), such methods comprising contacting T cells with an effective amount of N-acetylcysteine. In some of such embodiments the T cells are in vivo in a living subject, and the method comprises administering an effective amount of N-acetylcysteine to the living subject. In some such embodiments the subject is a human. In some of such embodiments, the methods further comprising contacting the T cells with an immune check point inhibitor, such as a PD1 inhibitor, a PDL1 inhibitor, or a CTLA4 inhibitor.

These and other aspects of the present invention are described further in the “Brief Description of the Figures,” “Figures,” “Examples,” and “Claims” sections of this patent application.

### BRIEF DESCRIPTION OF THE FIGURES

FIG. 1A-H. In vitro exhaustion in an antigen-specific mouse T-cell system. FIG. 1A. Schematic representation of in vitro antigen-specific T cell exhaustion model. OT-I ovalbumin-specific CD8+ T cells were isolated from mouse spleen and lymph nodes. They were then stimulated with SIINFEKL (OT-I-specific ovalbumin peptide) for 2 days using splenic dendritic cells as antigen presenting cells. Following 2-day stimulation, cells were co-cultured with B16 melanoma cells in the presence of absence of peptide for an additional 6 days. Phenotyping was performed using multi-color flow cytometry either with or without re-stimulation. Chronic stimulation induced T-cell exhaustion as noted by upregulation of inhibitory co-receptors PD-1 (FIG. 1B), PD-L1 (FIG. 1F), and LAG-3 (FIG. 1C), induction of the transcription factor Eomesodermin (FIG. 1G & H), and suppression of effector cytokine production (FIG. 1D & E).

FIG. 2A-B. In vitro exhaustion in an antigen-specific mouse T-cell system. FIG. 2A. Schematic representation of in vitro antigen-specific T cell exhaustion model. Phenotyping was performed using flow cytometry. Chronic stimulation induced T-cell exhaustion, as noted by upregulation of inhibitory co-receptor PD-1 and suppression of effector cytokine TNF-α production (FIG. 2B).

FIG. 3A-G. In vitro exhaustion in a polyclonal T-cell system. FIG. 3A. Schematic representation of in vitro T cell exhaustion model. Model can utilize mouse or human T cells. Data shown in FIG. 3B-E was generated using mouse T-cells obtained from a wild-type C57/BL6 mouse. Isolated CD3+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. Phenotyping was performed using multi-color flow cytometry either with or without re-stimulation. Chronic stimulation induced phenotypic and functional hallmarks of T-cell exhaustion, including expression of inhibitory co-receptors PD-1 (FIG. 3B) and LAG-3 (FIG. 3C) and suppression of cytokine production (FIGS. 3D & E). Similar results were obtained using human CD3+ T cells isolated from healthy donors (FIG. 3F-G). FIG. 3F. Schematic representation of in vitro polyclonal T cell exhaustion model using human CD3+ T cells isolated from healthy donors. Phenotyping was performed using flow cytometry. Chronic stimulation induced T-cell exhaustion, as noted by upregulation of inhibitory co-receptor PD-1 and suppression of effector cytokine TNF-α production (FIG. 3G).

FIG. 4A-C. In vitro exhausted T-cells are similar to tumor-infiltrating exhausted T-cells, T-cells from chronic viral infections, and immunotherapy non-responders. Isolated CD3+ T cells were stimulated acutely or chronically as in previous Figures. RNA-seq of chronic and acutely stimulated cells was then compared to expression profiles from exhausted tumor-infiltrating mouse T-cells (FIG. 4A), exhausted T-cells from mice with chronic LCMV infections (FIG. 4B), and isolated T-cells from patients who received checkpoint blockade with either anti-PD-1 or anti-CTLA-4 and failed to respond (FIG. 4C). The results demonstrate that in vitro exhausted T-cells are transcriptionally similar to T-cells isolated from both tumors and chronic viral infections, and importantly, effectively model T-cells from patients who do not respond to checkpoint blockade.

FIG. 5. Chronically stimulated T-cells do not kill. OT-I ovalbumin-specific CD8+ T cells were activated as in FIG. 1. After 8 days of acute or chronic stimulation, T-cells were co-cultured for 24 hours with B16 mouse melanoma cells that had been pulsed with SIINFEKL peptide and express a luciferase reporter. Killing capacity was quantitated as residual luminescence relative to un-pulsed B16 cells co-cultured with T-cells. The data in FIG. 5 demonstrated that chronically stimulated T-cells are unable to kill B16 melanoma cells.

FIG. 6A-B. Chronic TCR stimulation limits CD8 T-cell proliferation. FIG. 6A—schematic representation of model/method. This model can utilize mouse or human T cells. The data shown in FIG. 6B was generated using mouse CD8+T-cells obtained from a wild-type C57/BL6 mouse. Isolated CD8+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. Cells were counted every 2 days using a Coulter counter. The data in FIG. 6B demonstrates that chronic antigen stimulation limits T-cell proliferation. Similar results were obtained using human CD8+ T cells isolated from healthy donors.

FIG. 7A-B. Chronic TCR stimulation limits CD4 T-cell proliferation. Chronic TCR stimulation limits CD8 T-cell proliferation. FIG. 7A—schematic representation of model/method. This model can utilize mouse or human T cells. The data shown in FIG. 7B was generated using mouse CD4+ T-cells obtained from a wild-type C57/BL6 mouse. Isolated CD4+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. Cells were counted every 2 days using a Coulter counter. The data in FIG. 7B demonstrates that chronic antigen stimulation limits T-cell proliferation. Similar results were obtained using human CD4+ T cells isolated from healthy donors.

FIG. 8A-D. Chronic TCR stimulation induces persistent aerobic glycolysis. FIG. 8A—schematic representation of model/method. Isolated CD3+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. On Day 8, media was harvested and analyzed for glucose and lactate concentration using a YSI biochemical analyzer. Glucose and lactate consumption and excretion was calculated by comparing media from wells containing cells to media alone. Results demonstrate that chronically stimulated T-cells consume more glucose (FIG. 8B) and excrete more lactate per molecule of glucose consumed (FIG. 8C-D) than acutely stimulated T-cells.

FIG. 9A-D. Chronic TCR stimulation induces persistent aerobic glycolysis. FIG. 9A—schematic representation of model/method. Isolated CD3+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. On Day 8, cells were re-plated in 96-well plates and analyzed for oxygen consumption rate and extracellular acidification rate using a Seahorse 96-well extracellular flux analyzer. Results demonstrate that chronically stimulated T-cells demonstrate a higher extracellular acidification rate (FIG. 9C-D) and lower oxygen consumption rate (FIG. 9B, FIG. 9D) than acutely stimulated T-cells.

FIG. 10A-B. Metabolic isotope tracing using in vitro exhausted T-cells. Isolated CD3+ T cells were stimulated acutely or chronically as described (polyclonal model). Following 8 days of culture, T-cells were cultured in the presence of universally C13-labeled glucose (top) or glutamine (bottom) for 6 hours. Cells were fixed with ice-cold methanol, metabolites were extracted, derivatized, and analyzed by gas chromatography—mass spectrometry (GC-MS) as well as by liquid chromatography—mass spectrometry (LC-MS). The data in FIG. 10A-B shows that chronically stimulated T-cells show marked differences in labeling of tricarboxylic acid cycle intermediates as compared to conventionally stimulated T-cells. FIG. 10A and FIG. 10B provide data for different tricarboxylic acid cycle intermediates—as indicated on their respective horizontal axes.

FIG. 11A-B. Under conditions of high glucose availability, chronic T-cell stimulation induces oxidative stress. FIG. 11A—schematic representation of relevant metabolic pathways. Isolated CD8+ and CD4+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. On Day 8, chloromethyl 2′,7′-dichlorodihydrofluorescein diacetate (CM-DCFDA) was added to cells for 30 minutes prior to assessment of GFP fluorescence by flow cytometry. CM-DCFDA fluoresces in the GFP channel only when oxidized in the presence of reactive oxygen species (ROS) and therefore reflects the relative accumulation of ROS within cells. The results shown in FIG. 11B demonstrate that chronically (Ch) stimulated CD4+ and CD8+ T-cells exhibit increased accumulation of reactive oxygen species as compared to acutely stimulated (Ac) CD4+ and CD8+T-cells.

FIG. 12A-B. Under low glucose availability, chronically stimulated T-cells cannot survive. Isolated CD3+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days in media containing either 20 mM glucose, 2 mM glucose, or 0.5 mM glucose as indicated. Cells were counted every 2 days using a Coulter counter. The data presented in FIG. 12A-B demonstrates that chronically stimulated T-cells (FIG. 12A) show increasing susceptibility to low extracellular glucose availability and increased susceptibility to low extracellular glucose availability as compared to acutely stimulated T cells (FIG. 12A).

FIG. 13A-B. N-acetylcysteine restores proliferation of chronically stimulated T-cells. FIG. 13A—schematic representation of model/method. Isolated CD3+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days in the presence or absence of 10 mM N-acetylcysteine. Cells were counted every 2 days using a Coulter counter. The data presented in FIG. 13B demonstrates that N-acetylcysteine fully reverses the impairment in proliferation imposed by chronic stimulation.

FIG. 14A-D. N-acetylcysteine reverses endogenous T-cell exhaustion. Isolated CD3+ T cells were stimulated acutely or chronically as described elsewhere herein in the without anti-PD-L1 (FIG. 14A) or in presence or absence of anti-PD-L1 (FIG. 14B) and/or N-acetylcysteine (10 mM) (FIG. 14C). Following 8 days of culture, cells were re-stimulated for cytokine production (above) or co-cultured with tumor cells expressing specific antigen. Chronically stimulated T-cells showed reduced cytokine production (FIG. 14A) and were not able to kill tumor cells (FIG. 14D). This was minimally rescued by anti-PD-L1 therapy (FIG. 14B, FIG. 14D) but was effectively reversed by N-acetylcysteine (FIG. 14B, FIG. 14D).

FIG. 15A-C N-acetylcysteine reverses CAR-T cell exhaustion. Exhausted CAR-T cells were generated by transferring CD19-specific CAR-T cells into mice bearing CD19-expressing A20 lymphoma cells. After 14 days, CAR-T cells were isolated from mouse spleens and interrogated for proliferative capacity, oxidative stress, and cytokine production. Vehicle-treated exhausted CAR-T cells (pink) could not proliferate (FIG. 15A) or produce cytokine (FIG. 15C) and showed elevated levels of reactive oxygen species (FIG. 15B). N-acetylcysteine treatment (10 mM) enhanced CAR-T cell proliferation (FIG. 15A) and cytokine production (FIG. 15C) while reducing oxidative stress (FIG. 15B).

FIG. 16A-B. N-acetylcysteine restores T-cell memory. Isolated CD3+ T cells were stimulated acutely or chronically as described herein. Following 8 days of culture, RNA was extracted and analyzed for expression of the memory T-cell defining transcription factor Tcf7 N-acetylcysteine restored Tcf7 expression, which was markedly lost during chronic stimulation (FIG. 16A). Tumor-infiltrating T-cells from patients with melanoma who received checkpoint inhibitors were extracted and analyzed by single-cell RNA-seq. Gene expression analysis showed that induction of oxidative stress had a strong inverse correlation with generation of T-cell memory (FIG. 16B), consistent with the hypothesis that reversing oxidative stress with N-acetylcysteine can enhance T-cell immunotherapy by restoring memory T-cell differentiation.

FIG. 17A-C. xCT overexpression improves the anti-tumor function of T-cells. Isolated CD3+ T cells were stimulated acutely or chronically as described herein. Cells were retrovirally infected with either a control construct (EV) or a construct expressing the cysteine transporter xCT (FIG. 17A). Overexpression of xCT reversed the loss both T-cell growth (FIG. 17B) and cytokine production (FIG. 17C) during chronic stimulation.

FIG. 18A-C. Ophthalmate is a biomarker for oxidative stress in exhausted T-cells and is reversed by N-acetylcysteine. Isolated CD3+ T cells were stimulated acutely or chronically as described herein in the presence or absence of N-acetylcysteine (10 mM). After 8 days, cells were analyzed for intracellular ophthalmate levels by LC-MS. The schematic diagram in FIG. 18A demonstrates how ophthalmate is generated by enzymes that normally synthesize glutathione if glutathione is depleted and those enzymes are de-repressed. Ophthalmate levels were higher in chronically stimulated T-cells (FIG. 18B) and this was reversed by administration of N-acetylcysteine (FIG. 18C).

### DETAILED DESCRIPTION

The main embodiments of the present invention are described in the “Summary of the Invention,” “Brief Description of the Figures,” “Figures,” “Examples,” and “Claims” sections of this patent application. This Detailed Description section provides certain additional definitions and description relating to the invention and is intended to be read in conjunction with all other sections of the present patent application.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range.

As used herein, the terms “about” and “approximately,” when used in relation to numerical values, mean within + or −20% of the stated value.

As used herein the abbreviation “APC” refers to an Antigen Presenting Cell.

As used herein the abbreviation “IL-2” or “IL2” refers to interleukin 2.

As used herein the abbreviation “IFNγ” refers to interferon gamma.

As used herein the abbreviation “TNFα” refers to tumor necrosis factor alpha.

As used herein the abbreviation “LAG3” refers to lymphocyte-activation gene 3.

As used herein the abbreviation “PD-1” refers to Programmed Death 1, which is also known as Programmed Death Protein 1 or Programmed Cell Death Protein 1.

As used herein the abbreviation “PD-L1” refers to a ligand for PD-1.

As used herein the abbreviation “EOMES” refers to the transcription factor eomesodermin.

As used herein the term “antigen-specific,” when used in relation to T cells, refers to T cells that bind to a given antigen of interest. The term “antigen-specific” includes: (a) T cells that all have exactly the same T cell receptor (TCR)—i.e. “monoclonal antigen-specific T cells”, (b) T cells that have different TCRs but that bind to the same epitope/peptide of the given antigen of interest, and (c) T cells that have different TCRs and bind to different epitopes/peptides from within the given antigen of interest.

As used herein the term “polyclonal,” when used in relation to T cells, refers to a population of T cells having TCRs with differing antigen specificities—i.e. a population of T cells that is not specific to any particular antigen. Accordingly, the terms “polyclonal” and “non-antigen specific” may be used interchangeably herein in relation to T cells.

Several of the embodiments of the present invention involve antibodies. As used herein, the term “antibody” encompasses intact polyclonal antibodies, intact monoclonal antibodies, single-domain antibody, nanobody, antibody fragments (such as Fab, Fab′, F(ab′)2, and Fv fragments), single chain Fv (scFv) mutants, multi-specific antibodies such as bi-specific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. In some embodiments the antibody can be an immunoglobulin molecule of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked, or conjugated to other molecules such as toxins, radioisotopes, or any of the other specific molecules recited herein.

As used herein the term “effective amount” refers to an amount of an active agent as described herein that is sufficient to achieve, or contribute towards achieving, one or more desirable outcomes, such as those specifically described herein (e.g. reversal of T cell exhaustion). An appropriate “effective” amount in any individual case may be determined using standard techniques known in the art, such as dose escalation studies, and may be determined taking into account such factors as whether in vitro or in vivo use is desired, the desired route of administration (e.g. systemic vs. intratumoral), desired frequency of dosing, etc. Furthermore, an “effective amount” may be determined in the context of any co-administration method to be used. One of skill in the art can readily perform such dosing studies (whether using single agents or combinations of agents) to determine appropriate doses to use.

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In preferred embodiments the subjects are human.

When used herein, the term “substantially pure” refers to purity of greater than 75%, preferably greater than 80%, more preferably still greater than 90%, and most preferably greater than 95%.

The terms “inhibit,” “decrease,” “block,” and “suppress” and the like may be used interchangeably herein and refer to any detectable and statistically significant decrease in the specified parameter (e.g. the specified biological activity or expression of the specified marker), including full inhibition of the specified parameter. For example, the term “inhibition” (and interchangeable terms) can refer to a decrease of about 10%, 20%, 30%, 40%, 50%, 60%, 70%, 80%, 90% or 100% in the specified parameter. For example, when the term “decrease” is used to describe an effect on a marker of T cell exhaustion (such as PD-1 expression), the term may refer to any statistically significantly decrease that marker. The amount of the decrease may be determined relative to a suitable control—such as a control as described herein or a control as readily selected by one of ordinary skill in the art. In some embodiments, the terms “inhibit,” “decrease,” “block,” and “suppress” refer to a decrease in the specified parameter of at least 10%, or at least 20%, or at least 30%, or at least 40%, or at least 50%, or at least 60%, or at least 70%, or at least 80%, or at least 90% or about 100%, as determined, for example, using one of the assays described herein.

The terms “stimulate,” “increase,” “enhance,” “induce” and the like may be used interchangeably herein and refer to any detectable and statistically significant increase in the specified parameter (e.g. the specified biological activity or expression of the specified marker), including full stimulation of the specified parameter. For example, the term “increase” (and interchangeable terms) can refer to an increase of about 10%, 20%, 30%, 40%, 50%, 60%, 70%, 80%, 90% or 100% or of about 2-fold, 4-fold, 6-fold, 8-fold, 10-fold, or more, in the specified parameter. For example, when the term “increase” is used to describe an effect on a marker of T cell exhaustion (such as effector cytokine production), the term may refer to any statistically significantly increase that marker. The amount of the increase may be determined relative to a suitable control—such as a control as described herein or a control as readily selected by one of ordinary skill in the art. In some embodiments, the term “increase” (and interchangeable terms) refers to an increase in the specified parameter of at least 10%, or at least 20%, or at least 30%, or at least 40%, or at least 50%, or at least 60%, or at least 70%, or at least 80%, or at least 90% or at least 100%, or least 2-fold, or at least 4-fold, or at least 6-fold, or at least 8-fold, or at least 10-fold, in the specified parameter as determined, for example, using one of the assays described herein.

Other abbreviations and definitions are defined elsewhere in this patent specification, or else are used in accordance with their usual meaning in the art.

The T cells used as the starting point/input for the methods of inducing T cell exhaustion methods described herein can be any suitable T cells. In some embodiments T cells are not antigen-specific—i.e. they are polyclonal T cells. In some embodiments the T cells are antigen-specific T cells. In some embodiments the antigen-specific T cells bind to the same epitope/peptide of a given antigen of interest. In some embodiments the antigen-specific T cells are monoclonal antigen-specific T cells. In some embodiments the T cells are human T cells. In some embodiments the T cells are mouse T cells. In some embodiments the T cells are CD3+ T cells. In some embodiments the T cells are CD4+ T cells. In some embodiments the T cells are CD8+ T cells. In some embodiments the T cells are wild-type (not genetically engineered) T cells. In some embodiments the T cells are genetically engineered T cells. In some embodiments the T cells comprise a genetically engineered T cell receptor (TCR). In some embodiments the T cells are chimeric antigen receptor T cells (CAR T cells)—i.e. they comprise are chimeric antigen receptor.

The T cells used as the starting point/input for the methods of inducing T cell exhaustion methods described herein can be obtained from any suitable source. For example, in some embodiments the T cells may be cultured T cells (e.g. a T cell line). In some embodiments the T cells may be obtained from an animal. In some embodiments the T cells may be obtained from a mouse. In some embodiments the T cells may be obtained from a human. In some embodiments the T cells may be obtained from a blood sample. In some embodiments the T cells may be obtained from a donor blood sample. In some embodiments the T cells may be obtained from a sample of peripheral blood mononuclear cells. Methods of obtaining T cells from animals (including humans) are well known in the art. Similarly, methods of obtaining T cells having certain characteristics (e.g. CD4+ T cells or CD8+ T cells) are also well known in the art.

Several of the methods described herein involve “antigen presenting cells” (APCs). APCs are cells that can present peptides from an antigen on MHC molecules on their surface for recognition by T cells. In some embodiments any suitable APC may be used. In some embodiments the APCs are professional APCs that can present peptides on MHC II molecules on their surface. In some embodiments the professional APCs are dendritic cells, macrophages, or B cells. In some embodiments the APCs are non-professional APCs that can present peptides on MHC I molecules on their surface. In some embodiments the non-professional APCs are tumor cells or virus-infected cells. In many embodiments the term APCs is used to refer to professional APCs—as will be clear from the context in which the term is used. For example, in those embodiments that refer to using either: (a) APCs or (b) tumor cells or virus-infected cells, the APCs referred to are professional APCs—i.e. as distinguished from the tumor cells or virus infected cells, which are non-professional APCs.

The present invention can be further described and understood by reference to the following non-limiting “Examples.” It will be apparent to those skilled in the art that many modifications to the specific description provided in the Examples can be practiced without undue experimentation and without departing from the scope of the present invention and the present disclosure.

### EXAMPLES

These examples describe the development, characterization, and use of new methods for the induction and monitoring of T cell exhaustion in vitro. These new methods are effective in both human and non-human systems (e.g. using both human and non-human (e.g. mouse) T cells), and with both antigen-specific and polyclonal stimulation. The new methods described herein provide significant advantages over prior methods that require T cell exhaustion to be induced in vivo—such as in vivo methods that induce T cell exhaustion by chronic viral infection or adoptive transfer of T cells into tumor-bearing mice. Furthermore, the methods described herein offer a mechanism by which to test whether T cells can be re-invigorated with pharmacologic agents—which is demonstrated herein.

### Example 1

### Development and Characterization of In Vitro Models of T Cell Exhaustion

This Example describes the development and characterization of two in vitro models of T cell exhaustion—an antigen-specific T cell exhaustion model and a polyclonal T cell exhaustion model.

Antigen-Specific T cell Exhaustion Model

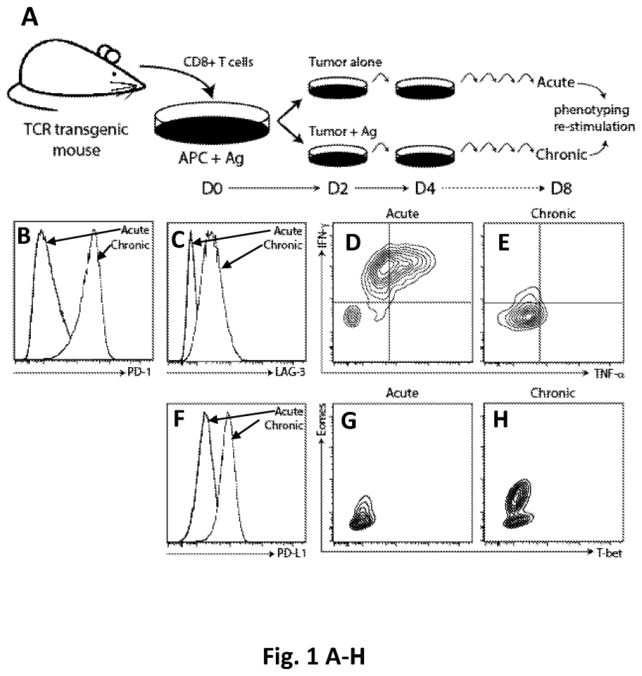

FIG. 1A provides a schematic representation of an antigen-specific T cell exhaustion model according to the present invention. The version of the model described in this example utilizes CD8+ T cells specific for an ovalbumin peptide. However, the model can also be used with T cells having different antigen specificities. In this example, spleen and lymph nodes containing CD8+ T cells specific for an ovalbumin peptide were isolated from an OT-I TCR transgenic mouse (https://www.jax.org/strain/003831). A single cell suspension was generated, red blood cells were lysed, and cells were incubated in vitro with 1 micromolar SIINFEKL (OT-I-specific ovalbumin peptide) for 2 days using splenic dendritic cells as antigen presenting cells in RPMI-1640 media containing fetal bovine serum, penicillin/streptomycin, L-glutamine, 2-mercaptoethanol, and 10 ng/mL recombinant IL-2 (referred to hereinafter as “T-cell media”). Following 2-days of stimulation, cells were harvested, centrifuged, and re-plated in T-cell media on 6-well plate containing B16-melanoma cells that had either been pulsed with 1 micromolar SIINFEKL or left un-pulsed. This procedure was repeated every 2 days, for a total of 8 days (3 total passages on days 2, 4, and 6); as a result, cells were in fresh media every 48 hours. Tumor cells were used for chronic stimulation in this assay to more fidelitously model the setting in which T-cells encounter persistent antigen during an anti-tumor response. B16 melanoma cells were used because they are derived from mice that share a background with OT-I T-cells (C57/B16 mice), therefore avoiding an allogeneic response, but similar results were obtained with other syngeneic tumors such as MC38 (colon) and EL4 (lymphoma) cell lines as well as other TCR transgenic T-cells, such as melanoma-specific PMEL CD8 TCR transgenic mice (https://www.jax.org/strain/005023). TCR transgenic mice were used because it allows stimulation of millions of T-cells at once in an identical fashion, which is useful for many of the assays described herein (such as metabolic assays). However, similar results were found with as few as 1000 antigen-specific T cells—i.e. numbers of T cells that can easily be isolated from non-transgenic mice and/or humans. The tumor antigen-specific model described here was used for CD8+ T-cells. However, it could be used for CD4+ T-cells provided that an MHC-II expressing cell line is used. We observed similar results with a D011.10 CD4 TCR transgenic mouse and RAW247 macrophages for chronic antigen presentation.

Polyclonal T Cell Exhaustion Model

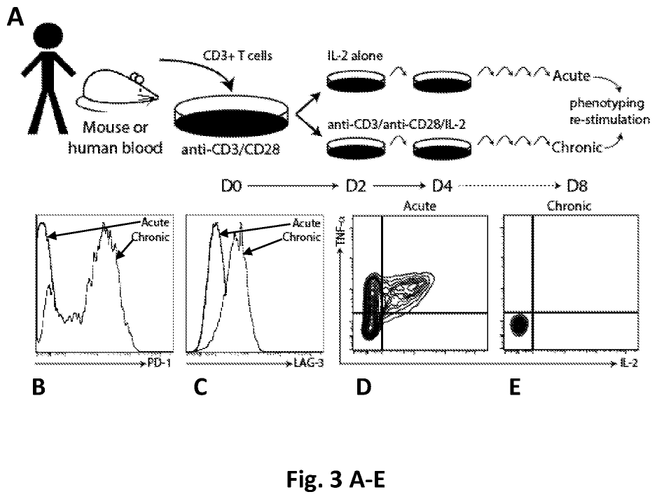

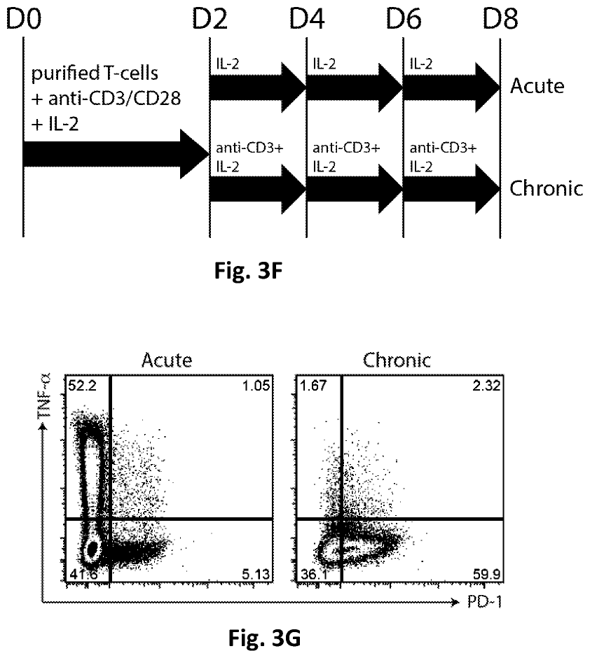

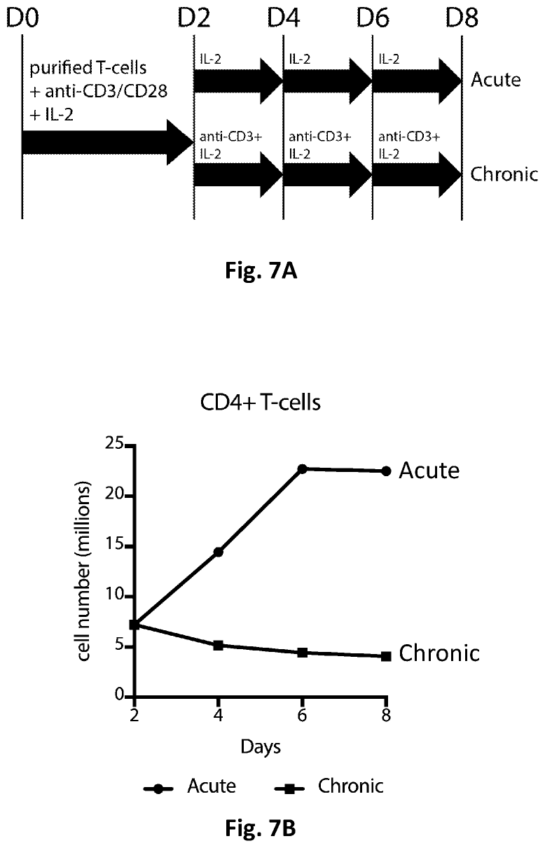

FIG. 3A and FIG. 3F provide schematic representations of a polyclonal T cell exhaustion model in which a generalized TCR and CD28 stimulus is used. Utilizing a generalized TCR and CD28 stimulus has multiple advantages. It allows for this assay to be applied to situations in which antigen-specific T-cells cannot be isolated, the specific antigen is unknown, or T-cells are responding to multiple antigens, as is frequently the case in anti-tumor immune responses. It can also easily be applied to both CD4+ and CD8+ T-cells and both mouse and human T-cells as shown in FIGS. 3, 6, and 7. In these figures, T-cells from the spleens of C57/B16 mice (FIG. 3A-E, 6 and 7) or healthy donor peripheral blood mononuclear cells (FIG. 3F-G) were isolated by negative selection using Untouched T-cell Dynabeads purification kits from Invitrogen. They were stimulated with 3 ug/mL plate-bound anti-CD3 (2C11, EBioscience) and 1 ug/mL plate-bound anti-CD28 (37.51, eBioscience) for 2 days in T-cell media containing IL-2 at 10 ng/mL. Following 2 days of stimulation, cells were either incubated in T-cell media containing IL-2 alone or serially re-plated on plates coated with anti-CD3 (3 ug/mL). This process was repeated with fresh media every 2 days for an additional 6 days (total of 8 days).

Characterization of T Cells Generated Using In Vitro T Cell Exhaustion Models

The phenotypes of the T cells generated using the models described above were characterized to assess known hallmarks of T cell exhaustion. For example, exhausted T cells are known to exhibit increased expression of several inhibitory receptors (such as PD-1, LAG-3, and PD-L1), decreased production of effector cytokines (such as IFNγ, TNFα and IL2), decreased proliferation rates, and decreased target cell killing activity (see, e.g., Wherry and Kurachi, 2015). In addition, enrichment of cells with specific transcription factor profiles, including high expression of eomesodermin (Eomes) and low expression of T-bet, has been linked to terminal exhaustion and poor disease control (Buggert et al., PLoS Pathogens, 17 Jul. 2014, 10(7):e1004251, and Li et al., Front Immunol. 2018 Dec. 18; 9:2981).

Expression Profiles of Factors Associated with T Cell Exhaustion

The expression of the inhibitory receptors PD-1, LAG-3, and PD-L1 and the effector cytokines IFNγ, TNFα and IL2 was analyzed by flow cytometry. Briefly, cells that were stimulated acutely or chronically for 8 days as described herein were re-stimulated after overnight rest using 50 ng/mL phorbol myristate acetate (PMA) and 500 ng/mL ionomycin. 90 minutes later, Brefeldin A was added to prevent cytokine release. 3 hours later cells were fixed and stained for surface markers as well as intracellular cytokines.

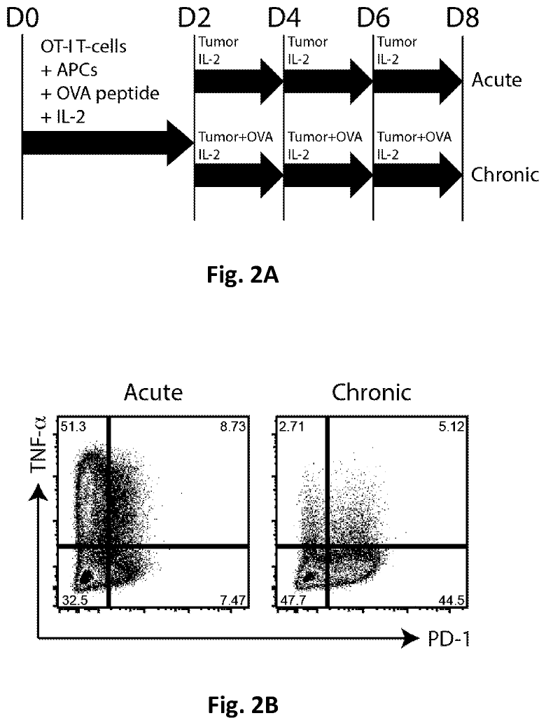

The results showed that chronic antigen-specific stimulation induced T-cell exhaustion as noted by upregulation of PD-1 (see FIG. 1B), PD-L1 (see FIG. 1F), and LAG-3 (see FIG. 1C), induction of the transcription factor EOMES (see FIGS. 1G & H), and suppression of effector cytokine production (see FIGS. 1D & E). Similar results are shown in FIG. 2, where chronic antigen-specific stimulation was shown to induce T-cell exhaustion as indicated by upregulation of PD-1 expression and suppression of TNF-α production (see FIG. 2B).

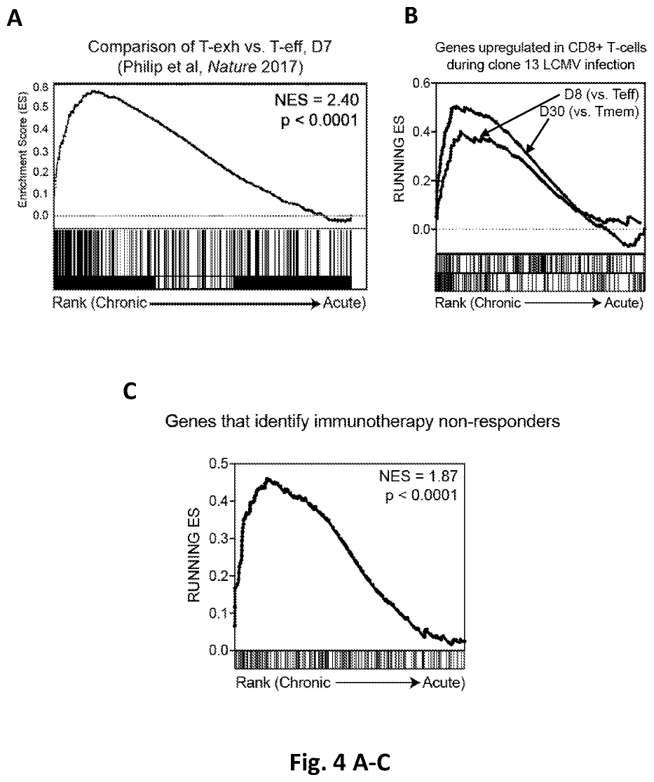

The results showed that chronic polyclonal stimulation also induced T-cell exhaustion as noted by increased expression of PD-1 (see FIG. 3B) and LAG-3 (see FIG. 3C) and suppression of cytokine production (see FIGS. 3D & E). Similar results are shown in FIG. 4 where chronic polyclonal stimulation was shown to induce T-cell exhaustion as indicated by upregulation of PD-1 expression and suppression of TNF-α production (FIG. 4B). While the results shown in FIG. 3 were generated by polyclonal stimulation of mouse T cells, similar results were also obtained when human CD3+T cells isolated from healthy donors were used in our polyclonal stimulation models (FIG. 4).

Cell Killing Activity

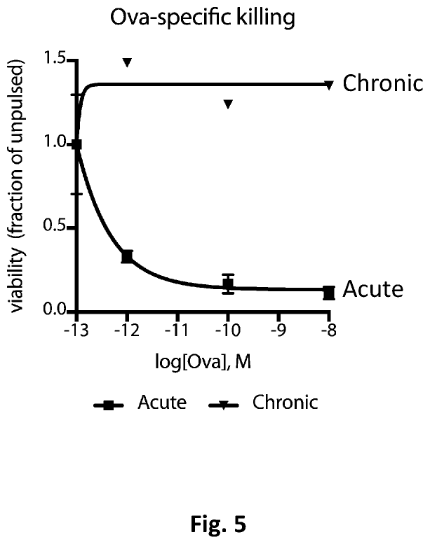

Ultimately, the hallmark of T-cell exhaustion in the context of both chronic viral infections and cancer requires a loss of effector function, defined as the absence of target cell killing. Therefore, it was imperative to establish that exhausted cells generated using this in vitro system were not able to kill target cells. To demonstrate this, OT-I TCR transgenic T-cells were stimulated as described herein. After 8 days of stimulation, T-cells were co-cultured with B16-melanoma cells expressing a luciferase reporter that had been pulsed with increasing doses of SIINFEKL peptide. 24 hours later luminescence was measured using a luminometer. Killing efficacy was determined by luminescence relative to B16 cells co-cultured with T-cells in the absence of specific peptide. The results in FIG. 5 demonstrate that in vitro exhausted T-cells generated using this system have indeed lost effector function.

T Cell Proliferation

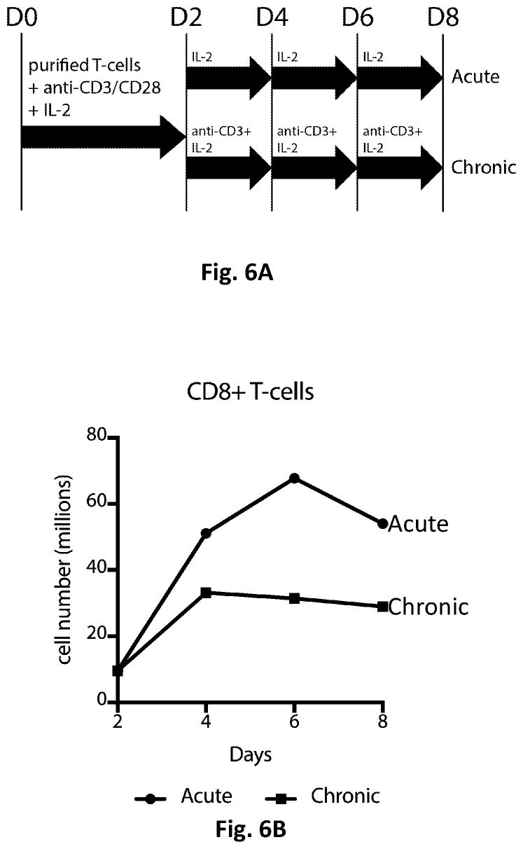

T-cell exhaustion is strongly associated with a loss of proliferative capacity (Barber et al, Nature 2006) and re-invigoration of exhausted T-cells is associated with a rapid increase in proliferation (Huang et al, Nature 2017). We therefore explored whether our in vitro exhaustion model limited T-cell proliferation. Isolated CD3+ T cells were stimulated for 2 days with plate-bound anti-CD3 and anti-CD28. Following 2-day stimulation, cells were either incubated in IL-2 alone or serially re-plated on plates coated with anti-CD3 and anti-CD28 every 2 days for an additional 6 days. Cells were counted every 2 days using a Coulter counter and clearly demonstrate that chronic antigen stimulation limits T-cell proliferation (FIG. 6B). Similar results were obtained using human CD3+ T cells isolated from healthy donors.

### Example 2

### Metabolic & Transcriptional Characterization

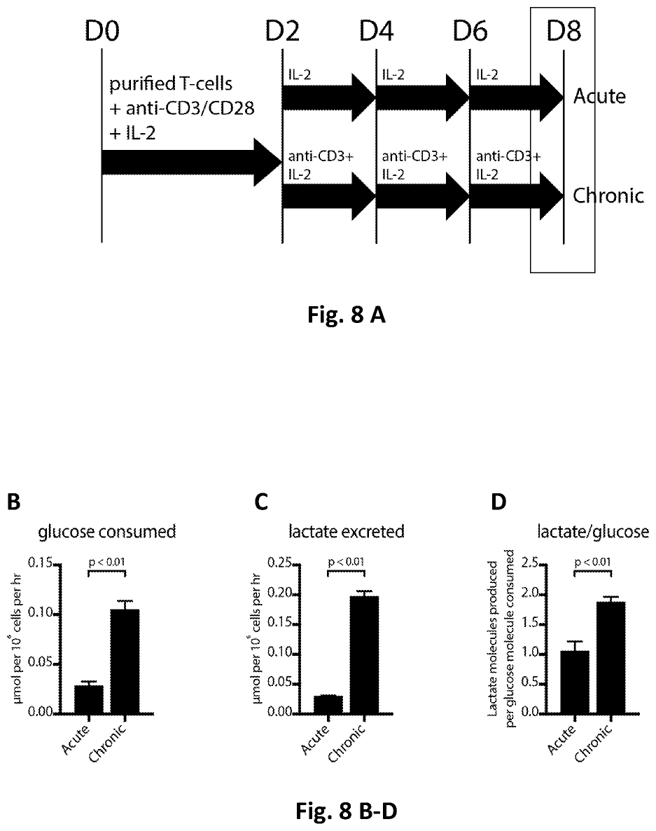

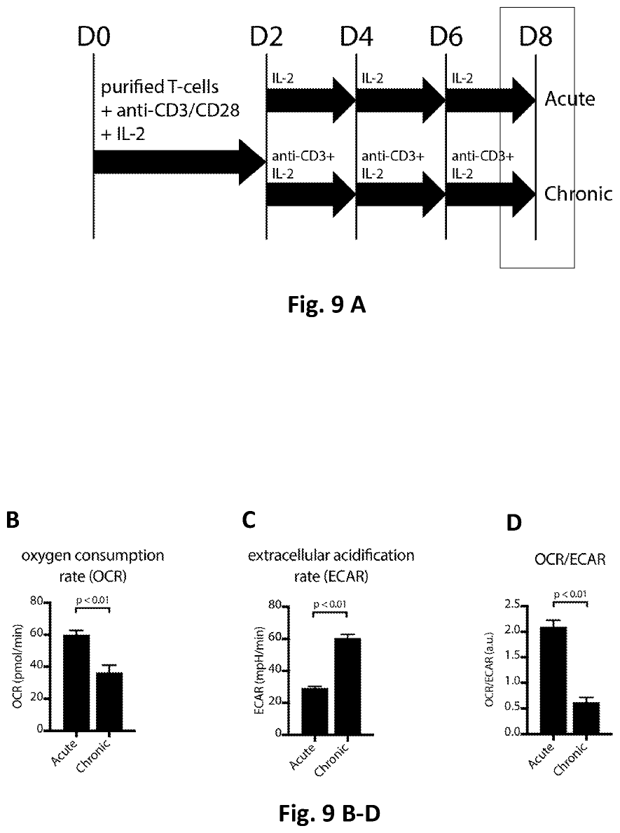

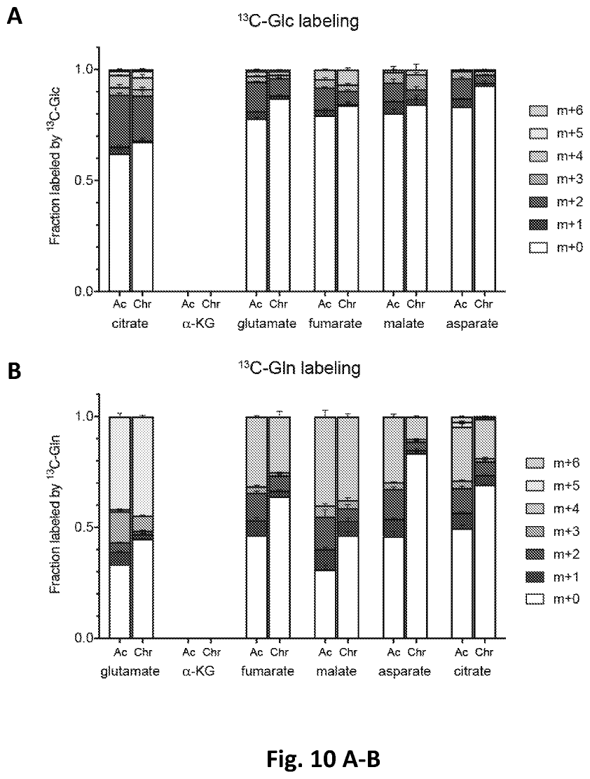

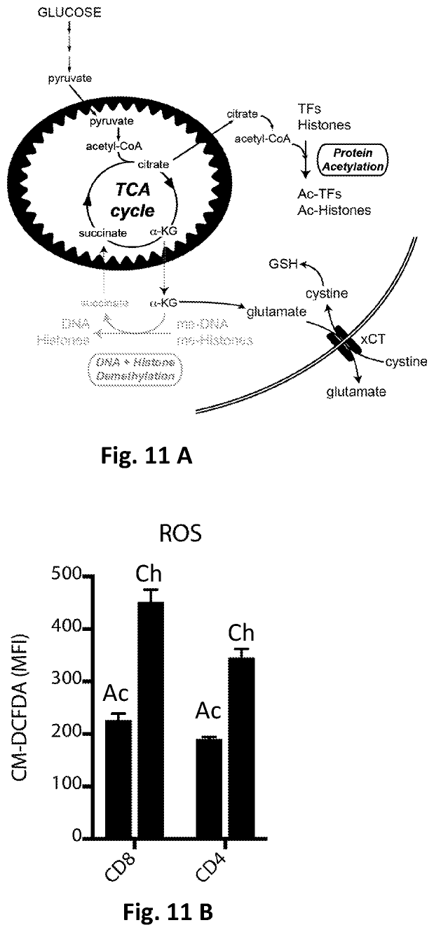

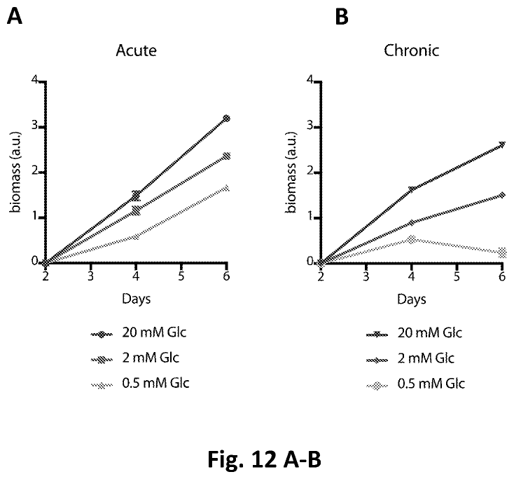

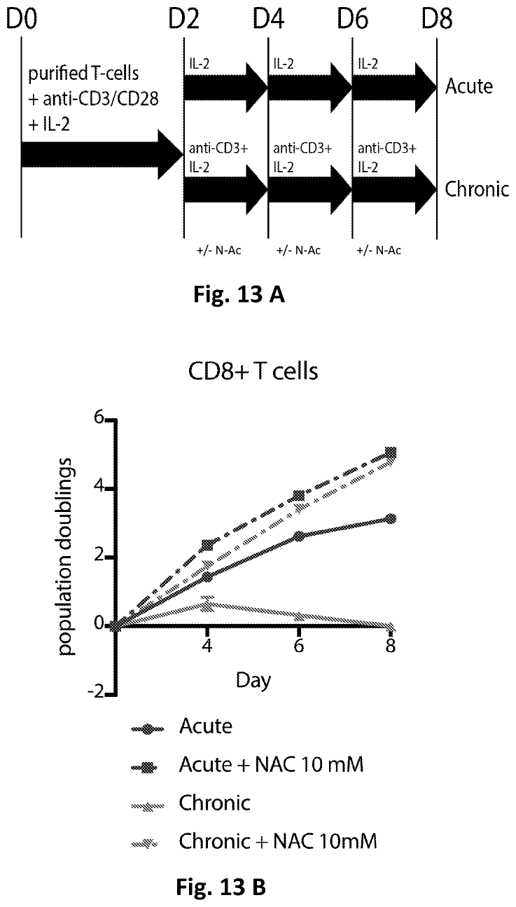

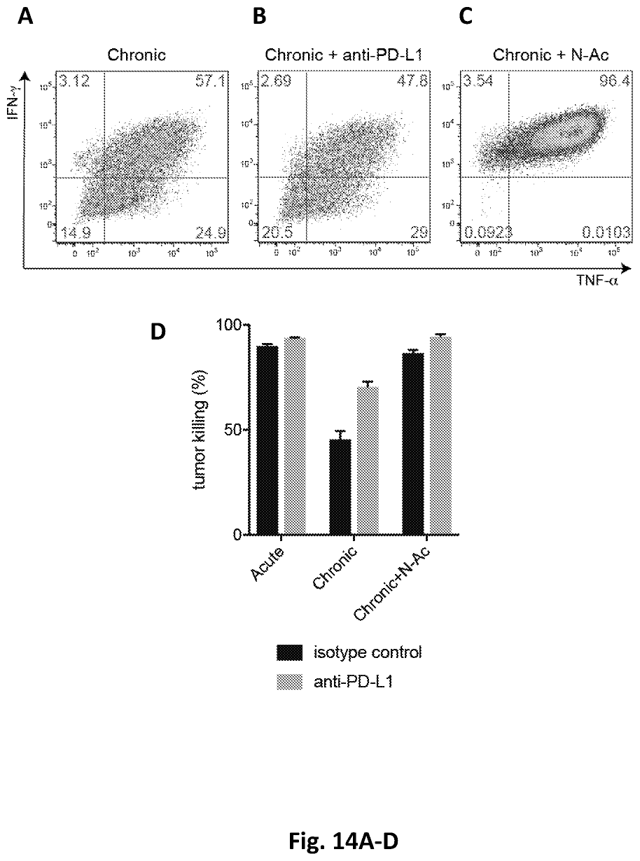

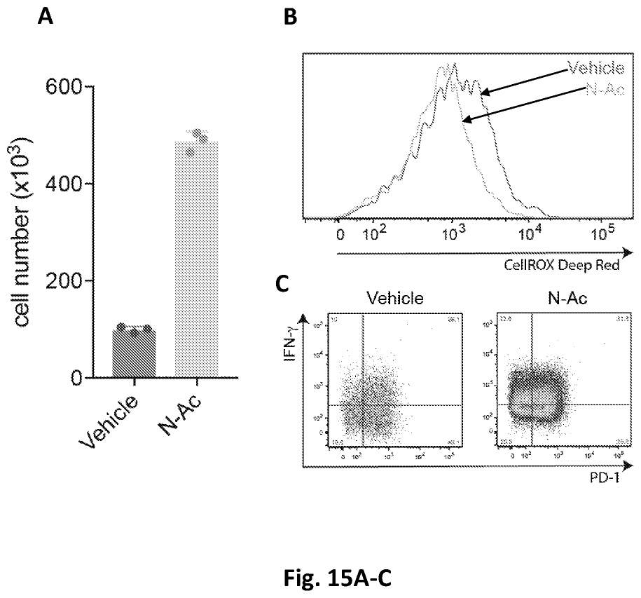

One of the most potent limitations of conventional in vivo systems for understanding T-cell exhaustion is the inability to understand changes in cellular metabolism associated with development of this pathology. This is for multiple reasons; the number of cells that can be isolated using these systems is limiting for mass spectrometry-based metabolic assays and the metabolism of cells changes dramatically during the process of flow cytometry-based sorting. As we demonstrate in FIG. 8-11, our in vitro system allows for in depth assessment of the metabolic behavior of T-cells during the development of T-cell exhaustion in a way that cannot be done using conventional systems. We are able to understand how nutrient utilization is radically altered during the development of T-cell exhaustion. These studies led to identification of oxidative stress as a metabolic hallmark of T-cell exhaustion and the development of N-acetylcysteine as a therapeutic intervention (FIG. 12-16). Therefore, this system allows for the discovery of novel metabolic alterations during the development of T-cell exhaustion that cannot be identified using conventional systems.

We also performed in-depth transcriptomic profiling of our in vitro system to ensure that it accurately recapiculates the process of T-cell exhaustion. As seen in FIG. 4 the transcriptomic profile of in vitro exhausted T-cells closely overlaps with both intratumoral signatures of T-cell exhaustion and T-cells from chronic viral infections, which are the two gold-standard in vivo models for generating exhausted T-cells. Moreover, an analysis of single-cell transcriptomic data from T-cells isolated from patients treated with checkpoint inhibitors shows that our in vitro system strongly correlates with the transcriptomic profile of T-cells from patients who did not respond to checkpoint blockade. This suggests that our in vitro system is not only an accurate model of T-cell exhaustion, but is actually a highly accurate model for checkpoint inhibitor-refractory T-cell exhaustion, which represents a true unmet need in immunotherapy.

### Example 3

### Reversal of Exhaustion by Pharmacologic Agents

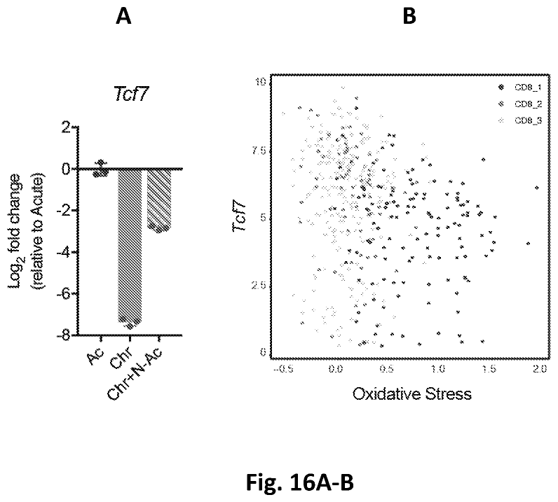

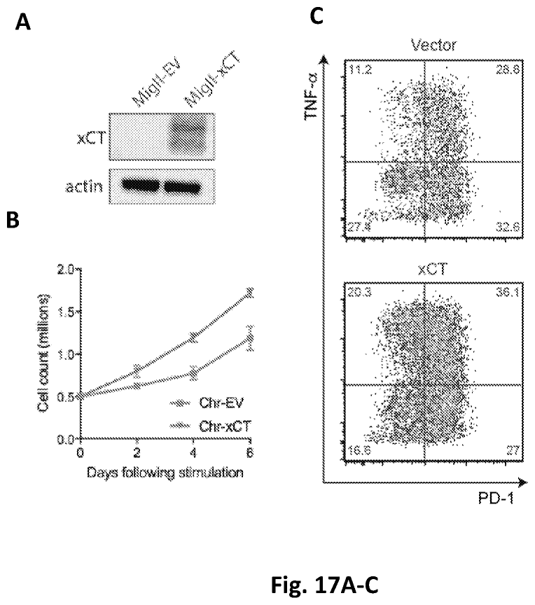

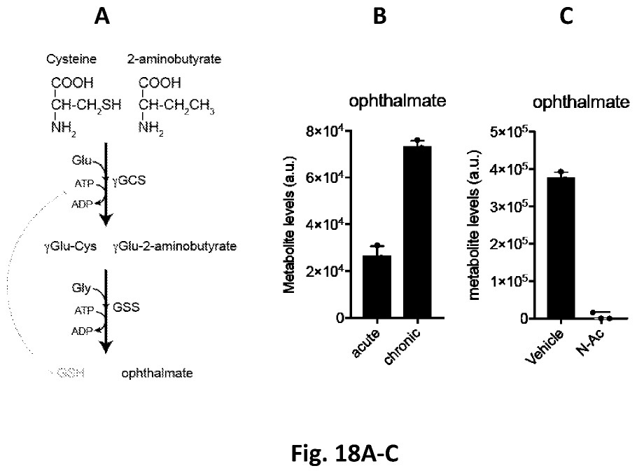

The in vitro system that developed and described herein allowed for metabolic evaluations that led directly to the identification of oxidative stress as a hallmark of T-cell exhaustion. We therefore evaluated whether adding N-acetylcysteine (10 mM) to T-cell media every 48 hours during days 2-8 of chronic stimulation would be sufficient to reverse the effects of chronic stimulation on T-cell growth and effector function. Indeed, we found that N-acetylcysteine was able to fully reverse the negative impact of chronic stimulation on T-cell proliferation and cytokine production; moreover, it fully restored target cell killing capacity and anti-PD-1 responsiveness. FIGS. 12-16. Moreover, this strategy was also effective in reversing the exhaustion of chimeric antigen receptor (CAR) T-cells. For this assay, T-cells retrovirally infected to express a chimeric antigen receptor targeting CD19 were adoptively transferred into mice who had previously received A20 B-cell lymphoma cells. This protocol is known to generate “exhausted” CAR T-cells by Day 14 following CAR T-cell transfer. We found that CAR T-cells isolated 14 days after transfer indeed had high levels of oxidative stress following re-stimulation with anti-CD3 and anti-CD28 that could be reversed by adding N-acetylcysteine (10 mM) to culture media; this allowed CAR T-cells to proliferate and produce cytokine. Additionally, N-acetylcysteine administration restored induction of the memory marker Tcf7, whose induction has been shown to be critical for checkpoint blockade efficacy. FIG. 16. To determine this, T-cells that had been acutely or chronically stimulated in the presence or absence of 10 mM N-acetylcysteine were assessed for Tcf7 expression via real-time quantitative PCR. The results clearly demonstrate that Tcf7 expression is suppressed by chronic stimulation and largely restored by the addition of N-acetylcysteine. This relationship is confirmed by an analysis of single-cell RNA-seq data from patients with melanoma treated with checkpoint inhibitors (Sade-Feldman Cell 2018), in whom genes associated with induction of oxidative stress negatively correlated with expression of Tcf7. Finally, the effects of N-acetylcysteine can be largely recapitulated by enabling cysteine uptake via retroviral overexpression of the xCT transporter. FIG. 17. Therefore, both pharmacologic and genetic approaches to reverse oxidative stress are able to largely reverse T-cell exhaustion.

We claim:

1. A method of inducing T cell exhaustion in vitro, the method comprising:
(a) culturing T cells in vitro for a first time-period under conditions that acutely stimulate T-cell receptor signaling, and
(b) subsequently culturing the T cells in vitro for a second time-period under conditions that chronically stimulate T-cell receptor signaling,
thereby generating exhausted T cells.

2. A method of inducing antigen-specific T cell exhaustion in vitro, the method comprising:
(a) culturing T cells that are specific for a given antigen with either: (i) the antigen and antigen-presenting cells, and optionally IL-2, or (ii) antigen-presenting cells that present the antigen, and optionally IL-2, wherein the culturing is performed in vitro the for a first time-period, and
(b) subsequently culturing the T cells with either: (i) the antigen and antigen-presenting cells, tumor cells, or virus-infected cells, and optionally IL-2, or (ii) antigen-presenting cells, tumor cells, or virus-infected cells that present the antigen on an MEW molecule, and optionally IL-2, wherein the culturing is performed in vitro for a second time-period,
thereby generating antigen-specific exhausted T cells.

3. A method of inducing polyclonal T cell exhaustion in vitro, the method comprising:
(a) culturing T cells with: (i) an anti-CD3 antibody and (ii) optionally an anti-CD28 antibody, and (iii) optionally IL-2, wherein the culturing is performed in vitro the for a first time-period, and
(b) subsequently culturing the T cells with: (i) an anti-CD3 antibody, (ii) optionally an anti-CD28 antibody, and (iii) optionally IL-2, wherein the culturing is performed in vitro the for a second time-period,
thereby generating polyclonal exhausted T cells.

4. The method of any of the preceding claims, wherein the exhausted T cells exhibit one or more of the following characteristics:
(a) increased expression of one of more of PD-1, LAG-3, and/or PD-L1, as compared to the starting T cells and/or non-exhausted T cells,
(b) decreased production of one or more effector cytokines as compared to the starting T cells and/or non-exhausted T cells,
(c) decreased proliferation as compared to the starting T cells and/or non-exhausted T cells, and
(d) decreased target cell killing activity as compared to the starting T cells and/or non-exhausted T cells.

5. The method of any of the preceding claims, further comprising performing an assay to measure one or more of the following characteristics in the starting T cells and/or or of the exhausted T cells:
(a) expression of PD-1, LAG-3, and/or PD-L1,
(b) production of one or more effector cytokines,
(c) proliferation rate, and
(d) target cell killing activity.

6. The method of any of claims 1-5, wherein the first time-period is about 1-3 days (24-72 hours).

7. The method of any of claims 1-5, wherein the first time-period is about 1-2 days (24-48 hours).

8. The method of any of claims 1-5, wherein the first time-period is about 1½-2 days (36-48 hours).

9. The method of any of claims 1-5, wherein the first time-period is about 2 days (48 hours).

10. The method of any of claims 1-5, wherein the second time-period is about 4-8 days (96-192 hours).

11. The method of claim any of claims 1-5, wherein the second time-period is about 6 days (144 hours).

12. The method of any of claims 1-5, wherein the second time-period is at least about 4 days (96 hours).

13. The method of any of claims 1-5, wherein the second time-period is at least about 6 days (144 hours).

14. The method of any of claims 1-5, wherein, during the second time-period, the T cells are cultured under conditions in which T-cell receptor signaling is chronically stimulated.

15. The method of claim 14, wherein the chronic stimulation is constant stimulation,

16. The method of claim 14, wherein the chronic stimulation is repetitive stimulation.

17. The method of any of claims 1-5, wherein, during the second time-period, the T cells are re-plated or split or passaged approximately every 1-2 days.

18. The method of any of claims 1-5, wherein, during the second time-period, the T cells are re-plated approximately every 1-2 days at a density of about 1 million cells per ml.

19. The method of any of claims 1-5, wherein, during the second time-period, fresh antigen or fresh an anti-CD3 and/or anti-CD28 antibody is added to the T-cell cultures approximately every 1-2 days.

20. The method of claim 1, wherein the T cells are polyclonal T cells and wherein T-cell receptor signaling is stimulated by contacting the T cells with an anti-CD3 antibody.

21. The method of claim 1, wherein the T cells are polyclonal T cells, and wherein T-cell receptor signaling is stimulated by contacting the T cells with (a) an anti-CD3 antibody, and (b) an anti-CD28 antibody.

22. The method of claim 1, wherein the T cells are antigen-specific T cells, and wherein T-cell receptor signaling is stimulated by contacting the T cells with the antigen for which the T cells are specific.

23. The method of claim 22, wherein the antigen is presented on the surface of an antigen-presenting cell, a tumor cell, or a virus-infected cell.

24. The method of claim 2 or claim 23, wherein the antigen-presenting cell is a professional antigen-presenting cell.

25. The method of claim 2 or claim 23, wherein the antigen-presenting cell is a dendritic cell.

26. The method of any of the preceding claims, wherein, during the second time-period, the T cells are cultured with tumor cells.

27. The method of any of the preceding claims, wherein the antigen is a tumor antigen, and wherein, during the second time-period, the T cells are cultured with tumor cells that express the tumor antigen.

28. The method of any of the preceding claims, wherein, during the second time-period, the T cells are cultured with virus-infected cells.

29. The method of any of the preceding claims, wherein the antigen in a viral antigen, and wherein, during the second time-period, the T cells are cultured with virus-infected cells that comprise the viral antigen.

30. The method of any of claims 3, 19, 20, and 21, wherein the an anti-CD3 antibody and/or the anti-CD28 antibody is bound to a solid support.

31. The method of claim 30, wherein the an anti-CD3 antibody and/or the anti-CD28 antibody is bound to a tissue culture plate or other tissue culture vessel.

32. The method of claim 30, wherein the an anti-CD3 antibody and/or the anti-CD28 antibody is bound to the surface of beads.

33. The method of any of the preceding claims, wherein the T cells are CD4+ T cells.

34. The method of any of the preceding claims, wherein the T cells are CD8+ T cells.

35. The method of any of the preceding claims, wherein the T cells are mammalian T cells.

36. The method of any of the preceding claims, wherein the T cells are rodent T cells.

37. The method of any of the preceding claims, wherein the T cells are mouse T cells.

38. The method of any of the preceding claims, wherein the T cells are primate T cells.

39. The method of any of the preceding claims, wherein the T cells are human T cells.

40. The method of claim 2, or any of the preceding claims that depends on claim 2, wherein the T cells are obtained from a TCR transgenic mouse.

41. The method of claim 1, claim 3, or any of the preceding claims that depends on claim 1 or claim 3, wherein the T cells are obtained from mammalian blood.

42. The method of claim 1, claim 3, or any of the preceding claims that depends on claim 1 or claim 3, wherein the T cells are obtained from human blood.

43. A population of exhausted T cells obtained using the method of any of the preceding claims.

44. A substantially purified population of exhausted T cells obtained using the method of any of the preceding claims.

45. A method of evaluating the effect of one more test agents on T cell exhaustion, the method comprising:
(a) performing the method of any of claims 1-42 in the presence of a test agent(s) and in the absence of the test agent(s), and
(b) performing an assay to measure one or more of the following characteristics in the T cells produced in step (a):
i. expression of PD-1, LAG-3, and/or PD-L1,
ii. production of one or more effector cytokines,
iii. proliferation rate,
iv. target cell killing activity,
wherein: (i) if the T cells produced in the presence of the test agent exhibit increased expression of PD-1, LAG-3, and/or PD-L1, decreased production of the one or more effector cytokines, a decreased proliferation rate, or decreased target cell killing activity as compared to the T cells produced in the absence of the test agent, the test agent increases T cell exhaustion, and wherein: (ii) if the T cells produced in the presence of the test agent exhibit decreased expression of PD-1, LAG-3, and/or PD-L1, increased production of the one or more effector cytokines, an increased proliferation rate, or increased target cell killing activity as compared to T cells produced in the absence of the test agent, the test agent decreases T cell exhaustion.

46. A method of evaluating the effect of one more test agents on reinvigoration of exhausted T cells, the method comprising:
(a) contacting a first population of exhausted T cells produced using the method of any of claims 1-42 with one or more test agents to produce a test population of T cells,
(b) contacting a second population of exhausted T cells produced using the method of any of claims 1-42 with either no test agent or with one or more control agents to produce a control population of T cells,
(c) performing an assay to measure one or more of the following characteristics in the test population of T cells and in the control population T cells:
i. expression of PD-1, LAG-3 and/or, PD-L1,
ii. production of one or more effector cytokines,
iii. proliferation rate, and
iv. target cell killing activity,
wherein: (i) if the test population of T cells exhibits increased expression of PD-1, LAG-3, and/or PD-L1, decreased production of the one or more effector cytokines, a decreased proliferation rate, or decreased target cell killing activity as compared to the control population of T cells, the test agent decreases T cell reinvigoration, and wherein: (ii) if the test population of T cells exhibits decreased expression of PD-1, LAG-3, and/or PD-L1, increased production of the one or more effector cytokines, an increased proliferation rate, or increased target cell killing activity as compared to the control population of T cells, the test agent increases T cell reinvigoration.

47. A method evaluating the T cell exhaustion status of patient-derived T cells, the method comprising: performing the method of claim 5, or any of the claims that depend on claim 5.

48. A method of re-invigorating exhausted T cells, the method comprising contacting the exhausted T cells with an effective amount of N-acetylcysteine.

49. A method of reducing or reversing T cell exhaustion, the method comprising contacting exhausted T cells with an effective amount of N-acetylcysteine.

50. A method of stimulating or increasing a T cell-mediated immune response, the method comprising contacting T cells with an effective amount of N-acetylcysteine.

51. A method of stimulating or increasing a T cell-mediated immune response to tumor cells, the method comprising contacting T cells with an effective amount of N-acetylcysteine.

52. A method of stimulating or increasing a T cell-mediated immune response to a chronic infection, the method comprising contacting T cells with an effective amount of N-acetylcysteine.

53. The method of any of claims 48-52 wherein the T cells are in vivo in a living subject, and wherein the method comprises administering an effective amount of the N-acetylcysteine to the living subject.

54. The method of any of claims 48-52 wherein the T cells are in a human subject and wherein the method comprises administering an effective amount of the N-acetylcysteine to the human subject.

55. The method of any of claims 48-54 further comprising contacting the T cells with an immune check point inhibitor.

56. The method of any of claims 48-54 further comprising contacting the T cells with a PD1 inhibitor, a PDL 1 inhibitor, or a CTLA4 inhibitor.

57. The method of claim 53 further comprising administering an effective amount of an immune check point inhibitor to the subject.

58. The method of claim 54 further comprising administering an effective amount of an immune check point inhibitor to the human subject.

59. A composition comprising N-acetylcysteine for use in a method according to any of claims 48-58.
